(19) United States
(12) Patent Application Publication
(10) Pub. No.: US 2003/0224462 A1
(43) Pub. Date: Dec. 4, 2003

(54) Immunoassay for cancer

(76) Inventor: George H. Parsons, Arlington, MA (US)

Correspondence Address: Ronald J. Baron, Esq., HOFFMANN & BARON, LLP, 6900 Jericho Turnpike, Syosset, NY, 11791, US

(21) Appl. No.: 10/162,133

(22) Filed: Jun. 4, 2002

(51) Int. Cl. G01N 33/574; C07K 16/30

(52) U.S. Cl. 435/7.23; 530/388.8

(57) ABSTRACT

The invention relates to new immunoassays using IgE type auto-antibodies specific for tumor antigens, especially prostate specific antigen (PSA). Such an assay can also be used to improve existing assays for PSA.

The invention relates to the field of immunoassays in cancer, especially prostate cancer.

Prostate-specific antigen (PSA) is a serine protease (more specifically a kallikrein) that has been discovered by Wang et al. (Invest. Urol. 17, 159-163, 1979) and as gamma-seminoprotein by Hara et al. (Jap. J. Legal Med 25, 322-324, 1971). Normal prostate epithelial cells and benign hyperplastic tissue produce more PSA than malignant prostatic tissue. The reason that PSA can be use as a tumor marker, though, lies in the fact that PSA is expressed in higher amounts in patients with prostate cancer due to the higher volume of tumor cells. Furthermore, it is detectable in blood serum because ‘leakage’ of the cells is promoted by tissue abnormalities within the diseased prostate that allow the enzyme to travel through the cell stoma into the bloodstream via capillaries and lymphatic pathways. The main known biological function of PSA is the cleavage of the major gel-forming proteins semenogelin and fibronectin to increase sperm motility.

Nowadays prostate cancer is diagnosed by measuring the amount of PSA in serum of patients through immunoassays. The commercial assays use different techniques to measure the PSA: some are immunoradiometric, some are enzyme immunoassays, some are chemiluminescent immunoassays. Most commonly these assays use monoclonal antibodies which have been derived from injecting animals with PSA.

Current assays for the diagnosis of prostate cancer detect the presence of the antigen PSA, which is also produced in healthy men with a benign hyperplasia. In Table 1 the PSA values for healthy subjects are given.

TABLE 1
PSA concentrations in the serum of normal, healthy men.

| Age in years | PSA concentration in serum |
|---|---|
| 40-49 | 0.0-2.5 ng/ml |
| 50-59 | 0.0-3.5 ng/ml |
| 60-69 | 0.0-4.5 ng/ml |
| 70-79 | 0.0-6.5 ng/ml |

In the early stages of prostate cancer the cancer cells produce PSA but the concentration of PSA in the serum does not exceed the normal background levels. In 1991, Lilja et al. (Clin. Chem. 37:1618-1625) discovered that PSA exists in more than one form circulating in serum. These forms are free PSA (PSA-f), bound PSA (PSA-ACT) and completed PSA (PSA-MG). Since the complexed PSA-MG is not immunoreactive the total levels of PSA in the blood is a combination of PSA-f and PSA-ACT Recent discoveries (reviewed by Stephan, C. et al., Urology 59, 2-8, 2002) have even complicated the field: PSA has been shown to bind or complex with α1-antichymotrypsin (PSA-ACT), with α1-protease inhibitor (PSA-API) and with α2-macroglobuin (PSA-A2M), while the free PSA can also be available in a precursor form (ProPSA). The free PSA (fPSA) represents on average only 5% to 35% of the total PSA (tPSA) concentration. The fPSA/tPSA ratio has been established as a clinical routine parameter since the mid-1990s, but the value can be influenced by factors such as prostate volume, race, sample stability, prostatic manipulations and drug history. Also, immunoassays suffer from interference, which can be caused by different phenomena. One reason for interference, which is recognised in immunoassays for the detection of thyroid hormone, is the existence of autoantibodies (Selby, C., Ann. Clin. Biochem. 36:704-721, 1999). Therefore, there is still need for an improved bioassay to increase the correct diagnosis of prostate cancer.

We now have found that an immunoassay in which autoantibodies to prostate specific antigen (PSA) are detected is suitable for the diagnosis of prostate cancer. As early as 1983 it has been established that IgG immunoglobulins specifically reacting with PSA could be detected in the serum of cancer patients (Chu, T. M. et al., Ann. NY Acad. Sci. 417:383-389, 1983). A recent study confirmed the existence of autoantibodies in prostate cancer patients (McNeel D. G. et al., J. Urol. 164(5): 1825-1829, 2000). An immunoassay for the detection of these autoantibodies would yield more reliable diagnostic results than the current immunoassay for the antigen. Such an immunoassay can be performed in any suitable way, e.g. by radioimmunoaasy (RIA), enzyme immunoassay (EIA, ELISA), fluorescence immunoassay and the like. The basic principles for devising such assays are well known to a person skilled in the art. Preferably in such an assay PSA is attached to a solid carrier (such as a magnetic particle or the surface of a 96-well plate) and a plasma sample from a patient suspected of prostate cancer is added. Any auto-antibody specific for PSA would bind to the PSA on the solid surface. After a wash to remove any unbound material, an antibody specific for human (auto-)antibodies and labelled (for instance with acridinium ester, AE) would be added, which would bind to the auto-antibody-PSA complex. After another wash to remove the unbound material, the label can be detected (for instance in case of AE by adding hydrogen peroxide which triggers the chemiluminescent light reaction from the AE). The amount of label detected is proportional to the amount of anti-PSA antibody in the sample and from this the concentration in the sample can be calculated.

Variations in the detection method of autoantibodies to PSA can be envisaged (such as using directly labelled PSA and an immobilised capture antibody). These variations are well known to a person skilled in the art and are thought to be embodied in the present invention.

We further discovered that specifically IgE type auto-antibodies are suitable as a marker for the detection of early stage prostate cancers. It is assumed that antibodies of the IgE type are the first to be formed by the immune system as a reaction to the growth of tumor cells and the concurring secretion of PSA into the serum. The hypothesis supporting this assumption is that PSA is a proteolytic enzyme, which, in general, are known to be involved in the cause of allergy, yielding the production of IgE type antibodies. One theory of allergy suggests that proteases from pollen, dust mite dung, dog and cat dander and various fruits like papaya and kiwi, stem from a misinterpretation of the proteolysis induced by these allergens on epithelial surfaces as an attack by a parasite. Parasites such as worms gain access to the host by proteolytically dissolving the epithelial barrier protecting the host.

Such an IgE response is triggered very early in the disease and the antibodies have a higher concentration and affinity then the later formed antibodies of the IgG and the IgM type.

An immunoassay for the detection of autoantibodies of the IgE type is essentially performed as described above, with the only difference being that the labelled antibody which reacts to the human auto-antibody must be specific for IgE type antibodies. Such specific antibodies are well known to a person skilled in the art and can be easily obtained.

It should also be noted that other human kallikreins (proteolytic enzymes such as PSA) are being researched as ovarian and other tumor markers. On basis of the above hypothesis such kallikreins would also be capable to cause an allergic reaction which would result in the formation of auto-antibodies of the IgE type. These auto-antibodies would then also be very useful for immunoassays for the detection of the malignacies to which the kallikreins are related.

Detection of auto-antibodies can not only be useful per se but may also provide a means to improve existing immunoassays. As mentioned above, one of the causes of interference in immunoassays is the existence of auto-antibodies. Specific detection of these antibodies could be used to correct the values measured of the antigen concentration, thereby improving the diagnostic result of the test. Since it has been discovered that complexes of PSA and immunoglobulin predominantly occur in cancer patients, this PSA should in fact be seen (and calculated) as free PSA. By correction for this phenomenon the fPSA/tPSA ratio would increase in such patients and thus a better diagnosis is warranted.

1. An immunoassay for use in the diagnosis of prostate cancer wherein autoantibodies to prostate specific antigen are detected.

2. An immunoassay according to claim 1 wherein the autoantibodies are of the IgE type.

3. An immunoassay according to claim 1 or 2 wherein the detection of autoantibodies to PSA is used to correct the measured value of the concentration of PSA in the serum.

4. A kit for performing an immunoassay according to any of claims 1-3.

5. An IgE type auto-antibody which is capable of binding to a tumor specific antigen.

6. An IgE type auto-antibody according to claim 5, which is capable of binding PSA.

7. An immunoassay in which an IgE type auto-antibody according to claim 5 is used.
